(19) United States
(12) Patent Application Publication
(10) Pub. No.: US 2023/0384319 A1
(43) Pub. Date: Nov. 30, 2023

(54) APPARATUS AND METHOD FOR DETECTING AN ANALYTE

(71) Applicant: SiPhox, Inc., Burlington, MA (US)

(72) Inventors: Michael Dubrovsky, Somerville, MA (US); Eric Hsu, Boston, MA (US); Elaine Taine, Columbia, SC (US); Xabier Arias, San Sebastian (ES); Yulia Rybakova, Arlington, MA (US)

(73) Assignee: SiPhox, Inc., Burlington, MA (US)

(21) Appl. No.: 18/199,171

(22) Filed: May 18, 2023

Related U.S. Application Data
(60) Provisional application No. 63/343,266, filed on May 18, 2022.

(51) Int. Cl. G01N 33/68; G01N 33/543; G01N 21/77

(52) U.S. Cl. CPC G01N 33/6854; G01N 33/54373; G01N 33/54326; G01N 21/77; G01N 2470/04; G01N 2800/24

(57) ABSTRACT

An apparatus for detecting an analyte, the apparatus includes a biosensor having at least a sensor surface functionalized with a binding ligand, wherein the at least a sensor surface is configured to selectively bind to an analyte, a microfluidic device configured to receive a sample fluid containing the analyte, incubate the biosensor with the sample fluid, and conjugate an anti-analyte molecule with a nanoparticle, wherein the nanoparticle is configured to provide a binding signal to the biosensor when the nanoparticle binds to the analyte bound to the binding ligand on the at least a sensor surface, and incubate the biosensor with the analyte bound to the binding ligand on the at least a sensor surface with the anti-analyte molecule conjugated with the nanoparticle, and a sensor circuit communicatively connected to the biosensor, wherein the sensor circuit is configured to detect at least an analyte characteristic of the analyte.

### CROSS-REFERENCE TO RELATED APPLICATIONS

This application claims the benefit of priority of U.S. Provisional Patent Application Ser. No. 63/343,266, filed on May 18, 2022, and entitled “IGE ISOLATION USING MAGNETIC BEADS,” the entirety of which is incorporated herein by reference.

### FIELD OF THE INVENTION

The present invention generally relates to the field of diagnostics and analyte detection. In particular, the present invention is directed to apparatus and method for detecting an analyte.

### BACKGROUND

The increase in allergy diagnoses in both adults and children is occurring at a staggering rate in the US. Currently, blood-based anti-allergen specific IgE (sIgE) test is one of the primary diagnostic methods. Serum proteins including anti-allergen IgG (sIgG) can bind to the capture-allergen functionalized on the sensor surface and compete with sIgE binding leading to false negative results. This effect is especially possible when low levels of allergen are present in the immunoassay.

### SUMMARY OF THE DISCLOSURE

In an aspect, an apparatus for detecting an analyte is described. The apparatus includes a biosensor, wherein the biosensor includes at least a sensor surface functionalized with a binding ligand, wherein the at least a sensor surface is configured to selectively bind to an analyte, a microfluidic device, wherein the microfluidic device is configured to receive a sample fluid containing the analyte, incubate the biosensor with the sample fluid, and conjugate an anti-analyte molecule with a nanoparticle, wherein the nanoparticle is configured to provide a binding signal to the biosensor when the nanoparticle binds to the analyte bound to the binding ligand on the at least a sensor surface, and incubate the biosensor with the analyte bound to the binding ligand on the at least a sensor surface with the anti-analyte molecule conjugated with the nanoparticle, and a sensor circuit communicatively connected to the biosensor, wherein the sensor circuit is configured to detect at least an analyte characteristic of the analyte.

In another aspect, a method for detecting an analyte is described. The method includes functionalizing, by a microfluidic device, at least a sensor surface of a biosensor with a binding ligand, wherein the at least a sensor surface is configured to selectively bind to an analyte, receiving, by the microfluidic device, a sample fluid containing the analyte, incubating, by the microfluidic device, the biosensor with the sample fluid, and conjugating, by the microfluidic device, an anti-analyte molecule with a nanoparticle, wherein the nanoparticle is configured to provide a binding signal to the biosensor when the nanoparticle binds to the analyte bound to the binding ligand on the at least a sensor surface, incubating, by the microfluidic device, the biosensor with the analyte bound to the binding ligand on the at least a sensor surface with the anti-analyte molecule conjugated with the nanoparticle, and detecting, by a sensor circuit communicatively connected to the biosensor, at least an analyte characteristic of the analyte.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram of an exemplary embodiment of an apparatus for detecting an analyte;

FIG. 2 is a diagram depicting an assay principle.

FIG. 3 is a diagram demonstrating an exemplary assay dynamic range.

FIG. 4 is a diagram demonstrating a signal decrease in presence of 50% serum.

FIG. 5 is a diagram showing that sIgG binding to the sensor surface perturbs sIgE quantification.

FIG. 6 is a diagram showing that IgE concentration using anti-IgE MNP noticeably improves sIgE detection.

FIG. 7 is a diagram showing an exemplary embodiment of a sandwich-type assay using magnetic beads;

FIG. 8 is a flow diagram of an exemplary method for detecting an analyte; and

FIG. 9 is a block diagram of a computing system that can be used to implement any one or more of the methodologies disclosed herein and any one or more portions thereof.

The drawings are not necessarily to scale and may be illustrated by phantom lines, diagrammatic representations, and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for detecting an analyte. In an embodiment, it involves creating an assay for IgE concentration from patient samples (blood, serum, plasma, and the like) to increase sensitivity of sIgE detection. Exemplary embodiments illustrating aspects of the present disclosure are described below in the context of several specific examples.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

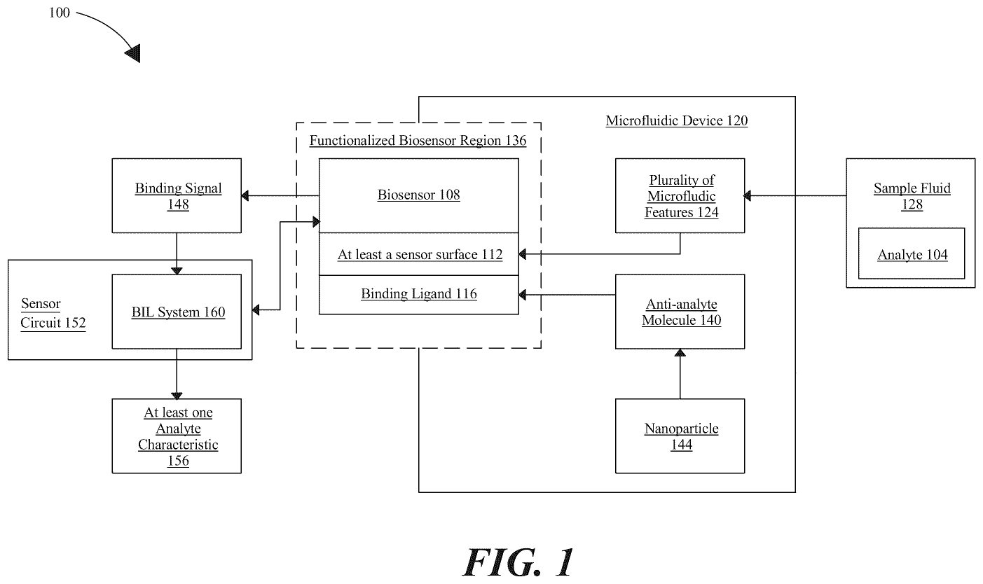

Now referring to FIG. 1, an exemplary embodiment of an apparatus 100 for detecting an analyte 104 is illustrated. An “analyte,” as described herein, is a substance that is of interest in an analytical procedure. As a non-limiting example, one or more analytes 104 may include glucose, proteins, hormones, antibodies, and the like. As another non-limiting example, the one or more analytes 108 may include Albumin, C-reactive protein, SARS-COV-2 protein, Thyroxine-binding globulin, Thyroxine-binding prealbumin (transthyretin,) Ceruloplasmin, Haptoglobin, Apolipoprotein A-I (protein of HDL,) Apolipoprotein A-II (another protein of HDL,) Apolipoprotein B-100 (protein of LDL,) Transferrin, Serum free light chains (info from LabCorp,) Antithrombin III, Fibrinogen, Lysozyme, Plasminogen, C3 complement, C4 complement, D-dimer, a1-Fetoprotein (AFP,) a2-Macroglobulin (AMG,) Retinol binding protein, Alpha1-Antitrypsin (A1AT or AAT,) a1-Acid Glycoprotein (or orosomucoid,) cxl-antichymotrypsin (Serpin family A member 3) ghrelin, Hemopexin, Complement factor H, Vitronectin, C4b binding protein (Complement component 4 binding protein beta,) Cysteine rich secretory glycoprotein LCCL domain containing 2 (Crispld2,) Complement C5, Alpha 1-B glycoprotein, Apolipoprotein H, Apolipoprotein A4, Plasminogen, GC vitamin D binding protein (DBP,) Histidine rich glycoprotein, Coagulation factor II, thrombin, Glycosylphosphatidylinositol specific phospholipase Dl, Complement Cls, Fetuin B, Kininogen 1, Complement C9, Gelsolin, Apolipoprotein C3, Serpin family A member 6, Apolipoprotein C1, Paraoxonase 1, Serum amyloid 4, Alpha-2 glycoprotein 1, zinc-binding, Afamin, Apolipoprotein C2, Clusterin, Apolipoprotein E, Serpin family A member 7, Complement component 4 binding protein alpha, Kallikrein Bl, Amyloid P component, Renalase, FAD dependent amine oxidase, Thrombospondin 1, Leucine rich alpha-2 glycoprotein 1, Lipopolysaccharide binding protein, Protein S, Retinal binding protein 4, Apolipoprotein F, Ficolin 3, Phospholipase transfer protein, Serpin family F member 1, Adiponectin, ClQ and collagen domain, Insulin such as growth factor binding acid labile subunit, Ficolin 2, Hyaluronan binding protein 2, Mannan binding lectin serine peptidase 1, C-type lectin domain family 3 member B, Coagulation factor V, Complement Clr subcomponent, Lecithin-cholesterol acyltransferase, CDS molecule, Serpin family A member 10, Apolipoprotein L1, Insulin like growth factor binding protein 3, Cholesterol ester transfer protein, CD14, Glutathione peroxidase 3, CD163, Paraoxanase 3, Protein Z, Ficolin 1, Transferrin receptor, ADAM metallopeptidase with thrombospondin type 1 motif 13, Complement factor D, Cystatin C, Apolipoprotein C4, Myeloperoxidase, Mannose binding lectin 2, Complement factor B, C-C motif chemokine ligand 28, Tenascin C, Vascular cell adhesion molecule 1 (VCAM1,) Cathelicidin antimicrobial peptide, Insulin like growth factor binding protein 2, Complement factor H related 3, Insulin like growth factor 2, Complement Clq C chain, Mannan binding lectin serine peptidase 2, Lipase G, Clq and TNF related 9, Fibrinogen alpha chain, Clq and TNF related 6, Von Willebrand factor, Gremlin 1, C1q and TNF related 5, C1q and TNF related 1, Serum amyloid Al, Angiogenin, C1q and TNF related 7, Orosomucoid 2, Angiopoietin like 3, Fc receptor like BMP4, Chromogranin A, and the like. Persons skilled in the art, upon reviewing the entirety of this disclosure, may appreciate various analytes 104 that may be used for an apparatus 100. Additional disclosures related to the at least an analyte 108 may be found in International Patent Application No PCT/US2022/037767, filed on Jul. 20, 2022, entitled as “WEARABLE BIOSENSORS FOR SEMI-INVASIVE, REAL-TIME MONITORING OF ANALYTES, AND RELATED METHODS AND APPARATUS,” the entirety of which is incorporated herein by reference.

With continued reference to FIG. 1, apparatus 100 includes a biosensor 108. As used in this disclosure, a “biosensor” is a sensor including a biological recognition component and a detector element. In some cases, the biological recognition component may include, without limitation, tissue, microorganisms, organelles, cell receptors, enzymes, antibodies, nucleic acids, and the like. Biological recognition component may interact with, bind with, or recognize an analyte such as, without limitation, any analyte listed above. In some cases, biological recognition component may be created using biological engineering. In some cases, biological recognition component may be chosen due to its favorable binding properties with the analyte that is sought to be detected. In an embodiment, biosensor 108 includes at least a sensor surface 112 functionalized with a binding ligand 116, wherein the at least a sensor surface 112 is configured to selectively bind to an analyte (i.e., a target analyte). As used in this disclosure, a “sensor surface” is a part of biosensor 108 that interacts with analyte or molecule being detected. In an embodiment, at least a sensor surface 112 may include a physical surface or interface of biosensor 108.

Still referring to FIG. 1, a “binding ligand,” for the purpose of this disclosure, is a substance that specifically interact with and binds to a complementary molecule or a molecular structure (i.e., a binding site) such as the biological recognition component. In some cases, the interaction between binding ligand 116 and the biological recognition component may be driven by one or more non-covalent forces such as, without limitation, hydrogen bonding, hydrophobic interactions, van der Waals forces, electrostatic forces, and/or the like. Such interaction may lead to a formation of a stable ligand-receptor complex. In some cases, binding ligand 116 may be small molecules, proteins nucleic acid, any other biological or chemical entities, and/or the like depending on target analyte. Additional disclosure related to the binding ligand disclosed herein may be found in International Patent Application No PCT/US2022/037767. Persons skilled in the art, upon reviewing the entirety of this disclosure, may appreciate various binding ligand 116 that may be used for apparatus 100.

In a non-limiting example, and with further reference to FIG. 1, a bioreceptor containing binding ligand 116 (i.e., biological recognition component) may be immobilized onto at least a sensor surface 112 through various chemical or physical methods, creating a functionalized sensor surface, wherein the functionalized sensor surface is configured to selectively bind target analyte while minimizing non-specific interactions with other components in a given sample. In an embodiment, functionalized sensor surface may be created via adsorption; for instance, and without limitation, apparatus may utilize weak forces such as non-covalent forces listed above to bind bioreceptor to the at least a sensor surface 112. It should be noted that adsorption is simple to perform but may result in a weak attachment, leading to potential loss of bioreceptors during further processing steps as described below. In another embodiment, functionalized sensor surface may be created via a covalent bonding, wherein the covalent bonding may involve forming strong and stable covalent bounds between bioreceptor and the at least a sensor surface 112; for instance, and without limitation, techniques including a use of silane chemistry for glass sensor surface, or self-assembled monolayer (SAM) with functional groups such as thiols, carboxyl's, amines, and the like for metal or polymer sensor surface. In a further embodiment, cross-linking may be used to chemically link bioreceptor to at least a sensor surface 112 with bifunctional or multifunctional reagents; for instance, and without limitation, cross-linking agent such as glutaraldehyde, carbodiimides (e.g., EDC/NHS), maleimides, and/or the like may be used to attach bioreceptor to at least a sensor surface 112. Other exemplary methods for immobilizing biological recognition element onto at least a sensor surface 112 such as, without limitation, entrapment and/or encapsulation, affinity-based immobilization, and/or the like may also be employed by apparatus 100.

With continued reference to FIG. 1, a “detector element,” for the purpose of this disclosure, is a device responsible for detecting a signal generated by the interaction of the analyte and the probe molecule (i.e., bioreceptor) immobilized on at least a sensor surface 112 of biosensor 108. Detector element may convert one form of energy or signal into another form of energy or signal, thereby enabling the detection, measurement, or monitoring of various physical, chemical, or biological phenomena. In a non-limiting example, detector element may include a transducer, wherein the transducer may receive an input signal in form of energy or a physical quantity such as light, sound, pressure, temperature, electrical signal or the like and covert the input signal into an output signal which may be of the same or different type of energy or physical quantity. Detector element may utilize optic, piezoelectric, electrochemical, electrochemiluminescence functions to produce output signal as a function of received input signal. In a non-limiting example, detector element of biosensor 108 may generate a signal resulting from the interaction of the analyte with the biological recognition component. Detector element may function in a physicochemical way to produce such signal. Exemplary embodiments of detector element may include, without limitation, electrochemical detector, optical detector, electronic detector, piezoelectric detector, gravimetric detector, pyroelectric detector, magnet detector, and the like.

With continued reference to FIG. 1, in an embodiment, biosensor 108 may include a photonic sensor chip. As used in this disclosure, “photonic sensor chip” a chip that includes electronic components that form a functional circuit that detects, generates, transports, and processes light. Additionally, and without limitation, the photonic sensor chip disclosed herein may be consistent with any photonic integrated circuit (PIC) found in International Patent Application No. PCT/US2022/037767, any sensor device found in U.S. patent application Ser. No. 18/121,712, filed on Mar. 15, 2023, entitled as “APPARATUS AND METHODS FOR PERFORMING MICROFLUIDIC-BASED BIOCHEMICAL ASSAYS,” having an attorney docket number of 1214-008USU1, any photonic sensor chip found in U.S. patent application Ser. No. 18/126,014, filed on Mar. 24, 2023, entitled as “PHOTONIC BIOSENSOR FOR MULTIPLEXED DIAGNOSTICS AND A METHOD OF USE,” the entirety of which are all incorporated herein by references.

With continued reference to FIG. 1, in some embodiments, biosensor 108 may include an optical waveguide. For the purposes of this disclosure, an “optical waveguide” is a structure that is designed to confine and guide electromagnetic waves along a path from one point to another. As a non-limiting example, electromagnetic waves may include ultraviolet, x-rays, gamma rays, infrared, microwave, radio waves, visible light, and the like. In some embodiments, the biosensor 108 may include a plurality of optical waveguide. In some embodiments, the optical waveguide may include dielectric materials, silicon, glass, polymer, semiconductor, and the like. In some embodiments, the optical waveguide may include various geometry of the waveguide. As a non-limiting example, the optical waveguide may include a straight waveguide, tapered waveguide, grating waveguide, and the like. In some embodiments, the optical waveguide may include various shapes, including but not limited to rectangular, circular, elliptical cross-sections, and the like. In some embodiments, the optical waveguide may include optical fiber waveguides, transparent dielectric waveguides, liquid light guides, liquid waveguides, light pipe, laser-inscribed waveguide, and the like. In some embodiments, the optical waveguide may include planar, strip, rib, fiber waveguides, and the like. In some embodiments, the optical waveguide may include single-mode, multi-mode, and the like. In some embodiments, the optical waveguide may include various refractive index distributions such as but not limited to step index distribution, gradient index distribution, and the like. For the purposes of this disclosure, “refractive index” of a material is a measure of how much the material can bend, or refract, light as it passes through it.

With continued reference to FIG. 1, in some embodiments, optical waveguide may include a first edge of the optical waveguide. For the purposes of this disclosure, an “first edge of an optical waveguide” is an edge of an optical waveguide that receives light wave from a source. As a non-limiting example, the first edge of an optical waveguide may receive the light wave (also called as an “optical signal,” “input optical signal,” “light signal” or “light”) directly from at least a light source as described below. As another non-limiting example, the first edge of an optical waveguide may receive the optical signal from a splitter network as described below. As another non-limiting example, the first edge of optical waveguide may receive the optical signal from a fiber-optic cable as described below. In some embodiments, a design and optimization of the first edge of an optical waveguides may depend on the mode profile, polarization state, refractive index of materials used, wavelength of the optical signal of an input source.

With continued reference to FIG. 1, in some embodiments, an optical waveguide may include a second edge of the optical waveguide. For the purposes of this disclosure, an “second edge of an optical waveguide” is an edge of an optical waveguide that guides an optical output out. For the purposes of this disclosure, an “optical output” is an optical signal that is output from the second edge of an optical waveguide. As a non-limiting example, the second edge of an optical waveguide may output the optical output to at least a photodetector as described below. As another non-limiting example, the second edge of an optical waveguide may output the optical output to the fiber-optic cable. In some embodiments, a design and optimization of the second edge of an optical waveguide may depend on the wavelength of the optical signal, polarization state, refractive index of materials used and/or mode profile of an output source.

With continued reference to FIG. 1, in some embodiments, biosensor 108 may include a splitter network. For the purposes of this disclosure, a “splitter network” is a device or a network that divides an input optical signal into two or more output optical signals. For the purposes of this disclosure, an “output optical signal” is an optical signal that is output from a splitter network. In an embodiment, each of the two or more output optical signals may include the same power as input power of the input optical signal. In another embodiment, each of two or more output optical signals may include a fraction of the input power of the input optical signal. In some embodiments, the two or more output optical signals may include a fixed power ratio determined by the design of the splitter network. For the purposes of this disclosure, “input power” of the input optical signal is amount of electromagnetic energy that is carried by an optical signal. The input power may be measured in units of watts. In some embodiments, a splitting ratio, the wavelength range, the insertion loss, and the polarization dependence may vary for the splitter network (also called as “splitter,” or “optical splitter.”) In a non-limiting example, splitter network may be used to split output signal from detector element into multiple pathways to a sensor circuit for further analysis and/or processing as described in further detail below.

With continued reference to FIG. 1, in some embodiments, a splitter network may include various types of splitter networks such as but not limited to Y-splitter, 1×N splitter, optical power splitter, fiber optic coupler, and the like. For the purposes of this disclosure, a “1×N splitter” is an optical splitter with one input port and multiple output ports. The 1×N splitter may be used to divide an input optical signal into multiple equal parts of two or more output optical signals. For example, and without limitation, a 1×2 optical splitter may split the input optical signal into two output optical signals, with each output optical signal having half the power of the input optical signal. For another example and without limitation, a 1×4 optical splitter may split the input optical signal into four output optical signals, with each output optical signal having a quarter of the power of the input optical signal. In some embodiments, the splitter network may be used in reverse as a combiner network. As a non-limiting example, the splitter network may combine multiple input optical signals into a single output optical signal.

With continued reference to FIG. 1, in an embodiment, a splitter network may include a passive optical splitter. For the purposes of this disclosure, a “passive optical splitter” is a type of optical splitter that does not require any external power or active electronic components to split an incoming optical signal. As a non-limiting example, the passive optical splitter may include Fused Biconical Taper (FBT) splitter, Planar Lightwave Circuit (PLC) splitter, Micro-Opto-Electro-Mechanical systems (MOEMS) splitter, Tapered Waveguide splitter, Fused Tapered couplers, Multimode Interference (MMI) splitter, and the like. In an embodiment, the passive optical splitter may be made by fusing and tapering two or more fibers together. In another embodiment, the passive optical splitter may be made by lithographically patterning a waveguide structure on a flat substrate such as but not limited to SOI substrate as described below. In some embodiments, the fibers or waveguides may be designed to split an input optical signal into two or more output optical signals with a predefined splitting ratio.

With continued reference to FIG. 1, in another embodiment, a splitter network may include an active optical splitter. For the purposes of this disclosure, an “active optical splitter” is a type of optical splitter that requires external power and active electronic components to split an incoming optical signal. As a non-limiting example, the active optical splitter may include semiconductor optical amplifiers (SOAs), electro-absorption modulators (EAMs), thermo-optic (TO) devices, and the like. In some embodiments, the active optical splitter may be integrated with a sensor circuit as described below. Splitter network described herein may include any splitter network disclosed in U.S. patent application Ser. No. 18/126,014.

With continued reference to FIG. 1, biosensor 108 may include one or more resonators. For the purposes of this disclosure, a “resonator” is a structure made of waveguide that can trap, store, transmit, process electromagnetic waves. In an embodiment, the one or more resonators may include photonic crystal cavities, grating structures, or interferometric structures such as Mach-Zehnder or Michelson interferometers, and the like. For the purposes of this disclosure, a “grating structure” is a structure of any regularly spaced collection of essentially identical, parallel, elongated elements, such as but not limited to optical waveguides. A “period A” of the grating determines the diffraction. As a non-limiting example, the grating structures may include a silicon sub-wavelength grating (SWG). For the purposes of this disclosure, a “sub-wavelength grating” is grating structures with a period A that is sufficiently small compared to the wavelength of light.

With continued reference to FIG. 1, in some embodiments, one or more resonators may include one or more ring resonators. For the purposes of this disclosure, a “ring resonator” is a waveguide that is a closed loop. In some embodiments, the one or more resonators may include various sizes and shapes of the loop (or a ring) and refractive index. In some embodiments, one or more ring resonators may be coupled with an optical waveguide. As a non-limiting example, one or more ring resonators may be in contact with the optical waveguide. As another non-limiting example, the one or more ring resonators may include a gap between the one or more ring resonators and the optical waveguide. In some embodiments, one or more ring resonators may use the principle of resonant wave coupling to filter or select certain wavelengths of light. In some embodiments, the biosensor 108 may include one or more arrays of one or more ring resonators. In an embodiment, each of the one or more ring resonators of biosensor 108 may detect the same one or more analytes 104 of a given sample. In another embodiment, each of the one or more ring resonators may detect different one or more analytes 104 of the given sample. As a non-limiting example, one ring resonator of the one or more ring resonators may detect SARS-COV-protein while another ring resonator of the one or more ring resonators detects glucose. When light is input into the loop (or the ring) of one or more ring resonators, the light may circulate around the loop multiple times due to total internal reflection, creating a standing wave pattern with constructive and/or destructive interference. Then, the one or more resonators may output an optical output. Because only a select few wavelengths are at resonance within the loop of the one or more ring resonators, the one or more ring resonators may function as a filter. In an embodiment, the light (or an input optical signal) may be input to a first edge of an optical waveguide, where the light may be from a fiber-optic cable with a PM fiber delivering the light from at least a light source. In another embodiment, two or more output optical signals (the light) from a splitter network that divide the input optical signal from the at least a light source may be input to the first edge of an optical waveguide. In another embodiment, an optical output may be output from a second edge of an optical waveguide to at least a photodetector. As a non-limiting example, the optical output may be output from the second edge of an optical waveguide to the at least a photodetector, then, to a reader device.

With continued reference to FIG. 1, in some embodiments, one or more ring resonators may include a single-ring resonator, double-ring resonator, add-drop filter, Vernier ring resonator, Bragg grating ring resonator, and the like. In some embodiments, one or more ring resonators may include a micro-ring resonator. For the purposes of this disclosure, a “micro-ring resonator” is a miniaturized version of the ring resonator. In some embodiments, the micro-ring resonator may be fabricated with a silicon or silicon-on-insulator (SOI) substrate using photolithography, etching, deposition, and/or other microfabrication techniques. For the purposes of this disclosure, “silicon-on-insulator substrate” is a type of semiconductor substrate. As a non-limiting example, the SOI substrate may include a thin layer of silicon, such as but not limited to silicon dioxide, on top of a layer of insulating material which is itself on top of a bulk silicon substrate. In some embodiments, the SOI substrate may reduce capacitance and parasitic effects, provide better isolation between devices, improve radiation hardness, and the like. In some embodiments, the SOI substrate may be fabricated for optical waveguides, ring resonators, splitter networks, and other photonic structures.

With continued reference to FIG. 1, in some embodiments, a biosensor 108 may utilize evanescent field of an optical waveguide and one or more resonators to probe properties and/or characteristics of the surrounding medium such as but not limited to one or more analytes 104 of sample fluid as described below. For the purposes of this disclosure, “evanescent field” is a type of electromagnetic field that exists outside the core of an optical waveguide. The evanescent field may decay exponentially with distance from the core and may carry less energy than the propagating mode inside the waveguide. When the waveguide and/or the one or more resonators is brought close to the one or more analytes 104, wherein the one or more analytes 104 are immobilized on the surface of the waveguide and/or the one or more resonators such as but not limited to with binding ligands 116 as described above, one or more characteristics of the one or more analytes 104 such as but not limited to their concentration, binding kinetics, conformational changes, or the like, may be probed using the evanescent field. Additional disclosure related to various methods to sense the one or more analytes may be found in International Patent Application No PCT/US2022/037767.

With continued reference to FIG. 1, in some embodiments, a biosensor 108 may include at least a photodetector. In some cases, the biosensor 108 may include a plurality of photodetectors, for instance a first photodetector and a second photodetector. In some cases, the first photodetector and/or the second photodetector may be configured to measure one or more of first optical output and second optical output, from a first waveguide and a second waveguide, respectively, such as but not limited to a second edge of an optical waveguide. The at least a first photodetector may be configured to convert the first optical output into a first sensor signal as a function of variance of an optical property of the first waveguide, where the first sensor signal may include without limitation any voltage and/or current waveform. Additionally, or alternatively, the biosensor 108 may include a second photodetector located down beam from the second waveguide. In some embodiments, the second photodetector may be configured to measure a variance of an optical property of second waveguide and convert the second optical output into a second sensor signal as a function of the variance of the optical property of the second waveguide.

With continued reference to FIG. 1, as used in this disclosure, a “photodetector” is any device that is sensitive to light and thereby able to detect light. In some cases, the at least a photodetector may include a photodiode, a photoresistor, a photosensor, a photovoltaic chip, and the like. In some cases, the at least a photodetector may include a Germanium-based photodiode. The at least a photodetector may include, without limitation, Avalanche Photodiodes (APDs), Single Photon Avalanche Diodes (SPADs), Silicon Photomultipliers (SiPMs), Photo-Multiplier Tubes (PMTs), Micro-Channel Plates (MCPs), Micro-Channel Plate Photomultiplier Tubes (MCP-PMTs), Indium gallium arsenide semiconductors (InGaAs), photodiodes, and/or photosensitive or photon-detecting circuit elements, semiconductors and/or transducers. “Avalanche Photo Diodes (APDs),” as used herein, are diodes (e.g., without limitation p-n, p-i-n, and others) reverse biased such that a single photon generated carrier can trigger a short, temporary “avalanche” of photocurrent on the order of milliamps or more caused by electrons being accelerated through a high field region of the diode and impact ionizing covalent bonds in the bulk material, these in turn triggering greater impact ionization of electron-hole pairs. APDs may provide a built-in stage of gain through avalanche multiplication. When the reverse bias is less than the breakdown voltage, the gain of the APD may be approximately linear. For silicon APDs, this gain may be on the order of 10-100. Material of APD may contribute to gains. Germanium APDs may detect infrared out to a wavelength of 1.7 micrometers. InGaAs may detect infrared out to a wavelength of 1.6 micrometers. Mercury Cadmium Telluride (HgCdTe) may detect infrared out to a wavelength of 14 micrometers. An APD reverse biased significantly above the breakdown voltage may be referred to as a Single Photon Avalanche Diode, or SPAD. In this case, the n-p electric field may be sufficiently high to sustain an avalanche of current with a single photon, hence referred to as “Geiger mode.” This avalanche current rises rapidly (sub-nanosecond), such that detection of the avalanche current can be used to approximate the arrival time of the incident photon. The SPAD may be pulled below breakdown voltage once triggered in order to reset or quench the avalanche current before another photon may be detected, as while the avalanche current is active carriers from additional photons may have a negligible effect on the current in the diode.

With continued reference to FIG. 1, in some cases, at least a photodetector may include a photosensor array, for example without limitation a one-dimensional array. The photosensor array may be configured to detect a variance in an optical property of waveguide. In some cases, first photodetector and/or second photodetector may be wavelength dependent. For instance, and without limitation, first photodetector and/or second photodetector may have a narrow range of wavelengths to which each of first photodetector and second photodetector are sensitive. As a further non-limiting example, each of first photodetector and second photodetector may be preceded by wavelength-specific optical filters such as bandpass filters and/or filter sets, or the like; in any case, a splitter may divide output from optical matrix multiplier as described below and provide it to each of first photodetector and second photodetector. Alternatively, or additionally, one or more optical elements may divide output from waveguide prior to provision to each of first photodetector and second photodetector, such that each of first photodetector and second photodetector receives a distinct wavelength and/or set of wavelengths. For example, and without limitation, in some cases a wavelength demultiplexer may be disposed between waveguides and first photodetector and/or second photodetector; and the wavelength demultiplexer may be configured to separate one or more lights or light arrays dependent upon wavelength. As used in this disclosure, a “wavelength demultiplexer” is a device that is configured to separate two or more wavelengths of light from a shared optical path. In some cases, a wavelength demultiplexer may include at least a dichroic beam splitter. In some cases, a wavelength demultiplexer may include any hot mirror, a cold mirror, a short-pass filter, a long pass filter, a notch filter, and the like. An exemplary wavelength demultiplexer may include part No. WDM-11P from OZ Optics of Ottawa, Ontario, Canada. Further examples of demultiplexers may include, without limitation, gratings, prisms, and/or any other devices and/or components for separating light by wavelengths that may occur to persons skilled in the art upon reviewing the entirety of this disclosure. In some cases, at least a photodetector may be communicative with sensor circuit, such that a sensed signal such as but not limited to one or more sensor signals may be communicated with sensor circuit.

With continued reference to FIG. 1, apparats 100 may include at least a light source. Apparatus 100 may be configured to provide an input optical signal using at least one light source. For the purposes of this disclosure, an “input optical signal” is an optical signal that includes electromagnetic radiation. In some embodiments, the input optical signal may be transmitted over optical fibers. As used in this disclosure, a “light source” is any device configured to emit electromagnetic radiation. As a non-limiting example, electromagnetic radiation may include ultraviolet (UV,) visible light, infrared light, and the like. At least a light source may control propagation, direction, polarization, intensity of light waves. In some embodiments, biosensor 108 may include lenses, mirrors, prisms, filters, optical fibers, and the like. In some embodiments, at least a light source may be tuned across the resonances of elements of biosensor 108. In some cases, at least a light source may include a coherent light source, which is configured to emit coherent light, for example a laser. In some cases, at least a light source may include a non-coherent light source configured to emit non-coherent light, for example a light emitting diode (LED). In some cases, at least a light source may emit a light having substantially one wavelength. In some cases, the at least a light source may emit the light having a wavelength range. The light may have a wavelength in an ultraviolet range, a visible range, a near-infrared range, a mid-infrared range, and/or a far-infrared range. For example, in some cases the light may have a wavelength within a range from about 100 nm to about 20 micrometers. In some cases, the light may have a wavelength within a range of about 400 nm to about 2,500 nm. The at least a light source may include, one or more diode lasers, which may be fabricated, without limitation, as an element of an integrated circuit; diode lasers may include, without limitation, a Fabry Perot cavity laser, which may have multiple modes permitting outputting light of multiple wavelengths, a quantum dot and/or quantum well-based Fabry Perot cavity laser, an external cavity laser, a mode-locked laser such as a gain-absorber system, configured to output light of multiple wavelengths, a distributed feedback (DFB) laser, a distributed Bragg reflector (DBR) laser, an optical frequency comb, and/or a vertical cavity surface emitting laser. At least a light source may additionally or alternatively include a light-emitting diode (LED), an organic LED (OLED) and/or any other light emitter. In some cases, at least a light source may be configured to couple light into biosensor 108 for instance into one or more waveguide described above.

With continued reference to FIG. 1, upon binding of analyte 104 to binding ligand 116 (i.e., bioreceptor) on at least a sensor surface 112 of biosensor 108, a detectable signal may be generated by the transduction mechanism employed by detector element of biosensor 108. Transduction mechanism may include, without limitation, electrical transduction (e.g., change in current, voltage, impedance, and/or the like), optical transduction (e.g., change in absorbance, fluorescence, luminescence, surface plasmon resonance (SPR), and/or any optical properties of biosensor 108), mechanical transduction (e.g., change in resonant frequency, mass, deflection, and/or any mechanical properties of biosensor 108), thermal transduction (e.g., change in temperature or heat flow of biosensor 108), and/or the like. In a non-limiting example, biosensor 108 may include a surface plasmon resonance (SPR) biosensor, wherein the SPR biosensor may utilize an optical transduction to produce a detectable signal. In some cases, SPR biosensor may exploit the changes in the local refractive index near at least a sensor surface 112 upon the binding of analyte 104 to bioreceptor immobilized on at least a sensor surface 112. light source such as, without limitation, a laser, or a broadband light source with a monochromator (i.e., an optical device used to select a narrow range of wavelength from a wider spectrum of light or radiation and delivers it to detector element) may be directed at the at least a sensor surface 112 at a specific angle, which excites the surface plasmons (i.e., collective oscillations of free elections at the interface between at least a sensor surface 112 and sample fluid 128) on at least a sensor surface 112. The reflected light from the at least a sensor surface may be collected by photodetector or a charge-coupled device (CCD) camera, and the intensity, angle, or wavelength of the reflected light may be measured. For example, and without limitation, when analyte 104 binds to bioreceptor on the at least a sensor surface 112, it may cause a change in the local refractive index near the at least a sensor surface 112. Such change in the refractive index may alter the conditions for the excitation of surface plasmons, resulting in a shift in the intensity, angle, or wavelength of the reflected light. The magnitude of this shift may be directly proportional to the amount of bound analyte 104 on the at least a sensor surface 112, allowing for the quantification of the analyte concentration in the fluid sample 128 in further processing steps as described below.

With continued reference to FIG. 1, as used in this disclosure, a “signal” is any intelligible representation of data, for example from one device to another. A signal may include an optical signal, a hydraulic signal, a pneumatic signal, a mechanical signal, an electric signal, a digital signal, an analog signal, and the like. In some cases, a signal may be used to communicate with a sensor circuit, for example by way of one or more ports. In some cases, a signal may be transmitted and/or received by a sensor circuit as described below, for example by way of an input/output port. An analog signal may be digitized, for example by way of an analog to digital converter. In some cases, an analog signal may be processed, for example by way of any analog signal processing steps described in this disclosure, prior to digitization. In some cases, a digital signal may be used to communicate between two or more devices, including without limitation sensor circuits. In some cases, a digital signal may be communicated by way of one or more communication protocols, including without limitation internet protocol (IP), controller area network (CAN) protocols, serial communication protocols (e.g., universal asynchronous receiver-transmitter [UART]), parallel communication protocols (e.g., IEEE 128 [printer port]), and the like.

With continued reference to FIG. 1, apparatus 100 includes a microfluidic device 120. As used in this disclosure, a “microfluidic device” is a device that is configured to act upon fluids at a small scale, such as without limitation a sub-millimeter scale. At small scales, surface forces may dominate volumetric forces. In a non-limiting example, microfluidic device may be consistent with any microfluidic device described in U.S. patent application Ser. No. 18/121,712, filed on Mar. 15, 2023, entitled “APPARATUS AND METHODS FOR PERFORMING MICROFLUIDIC-BASED BIOCHEMICAL ASSAYS,” the entirety of which is incorporated herein by reference.

With continued reference to FIG. 1, microfluidic device 120 may include a plurality of microfluidic features 124. As used in this disclosure, a “microfluidic feature” is a structure within microfluidic device 120 that is designed and/or configured to manipulate one or more fluids at micro scale. In a non-limiting example, plurality of microfluidic features 124 may include, without limitation, reservoir, microfluidic channel, conjugate pad, and the like as described in further detail below in this disclosure. In some cases, plurality of microfluidic feature 124 may enable a precise manipulation of fluids and samples in a controlled and/or reproducible manner within microfluidic device 120. In some embodiments, plurality of microfluidic features 124 of microfluidic device 120 may be designed and arranged based on particular needs of a given microfluidic-based biochemical assay. In other embodiments, plurality of microfluidic features 124 of microfluidic device 120 may be varied depending on the fluid, which is directly contact with plurality of microfluidic features 124. In a non-limiting example, attributes of plurality of microfluidic features 124 such as, without limitation, the size and/or shape of the substrate may be determined as a function of specific assay protocols. Exemplary embodiments of plurality of microfluidic features 124 are described in further detail below in this disclosure.

Still referring to FIG. 1, as used in this disclosure, a “microfluidic-based biochemical assay” is an assay on small volumes (i.e., in unit of ml or nl) of fluids. In some embodiments, microfluidic-based biochemical assay may be used for a wide range of applications, such as without limitation, medical diagnostics, drug discovery, environmental monitoring, and food safety testing, and the like. In a non-limiting example, apparatus 100 may be used to perform a microfluidic-based immunoassay for detecting the presence or quantity of a specific target molecule, such as, without limitation, IgE/sIgE. In microfluidic-based immunoassay, sample and reagents may be manipulated in plurality of microfluidic features such as, without limitation, plurality of microfluidic channels, which offers several advantages over traditional immunoassays including higher sensitivity, faster assay time, and lower sample and reagent consumption.

With continued reference to FIG. 1, microfluidic device 120 is configured to receive a fluid sample 128 containing analyte 104. As used in this disclosure, a “sample fluid” is a liquid specimen obtained from a living organism, environmental source, or otherwise a manufactured product, which is analyzed by apparatus 100 to detect, identify, or otherwise quantify specific components, such as, without limitation, analyte 104, biomolecules, microorganisms, and/or the like. In a non-limiting example, sample fluid 128 may include blood, plasma, urine, saliva, sweat, tears, cerebrospinal fluid for medical diagnostics from a patient.

With continued reference to FIG. 1, in some cases, plurality of microfluidic features 124 may include a reservoir configured to contain at least a fluid such as, without limitation, sample fluid 128. In an embodiment, reservoir may have at least an inlet, at least an outlet, or both. Reservoir may further include, without limitation, a well, a channel, a flow path, a flow cell, a pump, and the like. In a non-limiting example, sample fluid 128 received by microfluidic device 120 may be input through the at least an inlet into reservoir and/or output through the at least an outlet. At least an outlet may be connected to other components, devices, and/or microfluidic features within microfluidic device 104; for instance, and without limitation, at least an outlet may be connected to a microfluidic channel. Alternatively, or additionally, sample fluid 128 may include one or more suspensions and/or solutions of reagents, molecules, or other items to be analyzed and/or utilized, including without limitation monomers such as individual nucleotides, amino acids, or the like, one or more buffer solutions and/or saline solutions for rinsing steps. In a non-limiting example, receiving sample fluid 128 may include spiking a buffer fluid 132, wherein the buffer fluid 132 may include a PB S/EDTA/Tween-20 buffer. PB S/EDTA/Tween-20 buffer is a solution containing a mixture of three components” phosphate-buffered saline (PBS), ethylenediaminetetraacetic acid (EDTA), and Tween-20 detergent. In an embodiment, PBS is a buffer solution used to maintain the pH and osmolarity of the solution while EDTA is a chelating agent configured to bind to divalent cations such as calcium and magnesium to prevent their interference with the assay. Tween-20 is a detergent added to the solution configured to minimize non-specific binding and/or to increase the sensitivity of the assay. In a non-limiting example, spiking buffer fluid 132 may include diluting sample fluid 128.

With continued reference to FIG. 1, in some embodiments, microfluidic device 120 may include at least a flow component connected with at least a microfluidic feature of plurality of microfluidic features 124 configured to flow sample fluid 128 through biosensor 108. In some embodiments, at least a flow component may include a passive flow component configured to initiate a passive flow process. As used in this disclosure, a “passive flow component” is a component imparts a passive flow on at least a fluid, wherein the “passive flow,” for the purpose of this disclosure, is flow of the at least a fluid, which is induced absent any external actuators, fields, or power sources. As used in this disclosure, a “passive flow process” is a plurality of actions or steps taken on passive flow component in order to impart a passive flow on at least a fluid. The passive flow component may employ one or more passive flow techniques in order to initiate passive flow process; for instance, and without limitation, passive flow techniques may include osmosis, capillary action, surface tension, pressure, gravity-driven flow, hydrostatic flow, vacuums, and the like. The passive flow component may be in fluidic communication with the reservoir. The passive flow component may be configured to flow the sample fluid 128 contained in the reservoir with predetermined flow properties. As used in this disclosure, “flow properties” are characteristics related to a flow of a fluid. Exemplary non-limiting flow properties may include, without limitation, flow rate (in μl/min), flow velocity, integrated flow volume, pressure, differential pressure, and the like. In a non-limiting example, the passive flow component may be consistent with any passive flow component described in U.S. patent application Ser. No. 17/859,932, filed on Jul. 7, 2022, entitled “SYSTEM AND METHODS FOR FLUID SENSING USING PASSIVE FLOW,” the entirety of which is incorporated herein by reference.

With continued reference to FIG. 1, in other embodiments, at least a flow component may include an active flow component configured to initiate an active flow process. As used in this disclosure, an “active flow component” is a component that imparts an active flow on at least a fluid, wherein the “active flow,” for the purpose of this disclosure, is flow of the at least a fluid which is induced by external actuators, fields, or power sources. As used in this disclosure, an “active flow process” is a plurality of actions or steps taken on active flow component in order to impart active flow on at least a fluid. In some embodiments, the active flow component is in fluidic communication with the reservoir. In a non-limiting example, the active flow component may include one or more pumps. The one or more pumps may include a substantially constant pressure pump (e.g., centrifugal pump) or a substantially constant flow pump (e.g., positive displacement pump, gear pump, and the like). The one or more pumps can be hydrostatic or hydrodynamic. As used in this disclosure, a “pump” is a mechanical source of power that converts mechanical power into fluidic energy. The one or more pumps may generate flow with enough power to overcome pressure induced by a load at a pump outlet. The one or more pumps may generate a vacuum at a pump inlet, thereby forcing sample fluid 128 from at least a reservoir into the pump inlet to the one or more pumps pump and by mechanical action delivering sample fluid 128 to a pump outlet. The hydrostatic pumps may include positive displacement pumps. The hydrodynamic pumps can be fixed displacement pumps, in which displacement may not be adjusted, or variable displacement pumps, in which the displacement may be adjusted. Exemplary non-limiting pumps include gear pumps, rotary vane pumps, screw pumps, bent axis pumps, inline axial piston pumps, radial piston pumps, and the like. The one or more pumps may be powered by any rotational mechanical work source, for example without limitation and electric motor or a power take off from an engine. The one or more pumps may be in fluidic communication with at least a reservoir. In some cases, at least a reservoir may be unpressurized and/or vented. In a non-limiting example, the active flow component may be consistent with any active flow component described in U.S. patent application Ser. No. 18/107,135, filed on Feb. 8, 2023, entitled “APPARATUS AND METHODS FOR ACTUATING FLUIDS IN A BIOSENSOR CARTRIDGE,” the entirety of which is incorporated herein by reference.

With continued reference to FIG. 1, microfluidic device 120 is configured to incubate biosensor 108 with sample fluid 128. In some cases, microfluidic device 120 may facilitate the interaction between analyte 104 and binding ligand 116 immobilized on at least a sensor surface 112, leading to the formation of stable and specific complexes via an incubation process. In some cases, the incubation temperature, and conditions of buffer fluid 132, such as pH, ionic strength, the presence of additives, and/or the like may influence the binding kinetics (i.e., rate and strength of binding interactions between two or more molecules) and the stability of the analyte-bioreceptor complexes. In an embodiment, during this incubation process, sample fluid 128, containing the analyte 104 of interest, may be introduced into microfluidic device 120 and directed from reservoir towards a functionalized biosensor region 136 via flow component as described above. As used in this disclosure, a “functionalized biosensor region” refers to a specific area within microfluidic device 120 configured to place biosensor 108 with at least a sensor surface 112 functionalized with binding ligand 116. In a non-limiting example, biosensor 108 may be integrated into microfluidic device 120. Functionalized biosensor region 136 may match with at least a surface 112 of biosensor 108. In this case, Functionalized biosensor region 138 may be defined by at least an alignment feature of microfluidic device 120. An “alignment feature,” for the purpose of this disclosure, is a physical feature that helps to precisely align components of microfluidic device 120 with other components. Alignment feature may be configured for precise positioning and attaching biosensor 108. In some embodiments, alignment feature may be configured for precise positioning and attaching other components external to apparatus 100; for instance, and without limitation, without limitation, an external device such as a sensor circuit (i.e., an external reader) may be coupled with apparatus 100 through one or more alignment features such as, without limitation, a multi-fiber push connector (MPO), bracket, press fastener (with spring mechanism) and the like. In other cases, alignment feature may be configured for precise positioning plurality of microfluidic feature 124; for instance, and without limitation, microfluidic channel of microfluidic device 120 may be etched along alignment feature. Alignment feature described herein may be consistent with any alignment feature disclosed in U.S. patent application Ser. No. 18/121,712.

In a non-limiting example, and still referring to FIG. 1, alignment feature may include a slightly depressed plane, wherein the slightly depressed plane may include a same surface area with at least a surface 112 of biosensor 108 functionalized with binding ligand 116. In some embodiments, microfluidic feature may be connected to alignment feature, allowing sample fluid 128 to flow into the depressed plane containing biosensor 108 and contact at least a sensor surface 112 of biosensor 108. Microfluidic feature and/or alignment feature may ensure a precise control over the flow of sample fluid 128 and enabling the manipulation of minute sample fluid volumes. Functionalized biosensor region 136 may provide optimal conditions for analyte-bioreceptor interaction. In some cases, microfluidic device 120 may be configured to incubate biosensor 108 with sample fluid 128 at functionalized biosensor region 136. Such incubation process may be governed by a plurality of kinetic factors, such as, without limitation, mass transport, reaction rates, and/or the like. In a non-limiting example, mass transport of analyte 104 to at least a sensor surface 112 of biosensor 108 may occur via diffusion, convection, or a combination of both mechanisms. In some cases, reaction rate may refer to the association and dissociation kinetics between analyte 104 and binding ligand 116, which depend on their intrinsic binding properties, such as, without limitation, affinity, specificity, and the like.

With continued reference to FIG. 1, microfluidic device 120 is configured to conjugate an anti-analyte molecule 140 with a nanoparticle 144. As used in this disclosure, an “anti-analyte molecule” is a specific type of binding ligand 116 (i.e., bioreceptor) that selectively recognizes and binds to target analyte 104. In some cases, anti-analyte molecule may be biological in nature; for instance, and without limitation, anti-analyte molecule may include antibody, aptamer, any other biomolecule that exhibit high specificity and affinity for target analyte 104, or the like. High affinity ensures that the anti-analyte molecule can bind to target analyte 104 with strong binding forces, while high specificity and selectivity ensure minimal cross-reactivity with other components in sample fluid 128. In an embodiment, anti-analyte molecule 140 may include antibody (i.e., a protein produced by the immune system in response to the presence of a foreign substance). In a non-limiting example, antibody may exhibit high specificity and affinity for a target antigen such as a specific allergen; therefore, antibody is an ideal bioreceptors for analyte detection. Biosensor 108 may include at least a sensor surface 112 functionalized with AraH2 protein (i.e., a major peanut allergen) may be incubated, using microfluidic device 120, with a sample fluid 128 containing a plurality of anti-AraH2 antibodies, which bind to the at least a sensor surface 112 of biosensor 108. In another non-limiting example, anti-analyte molecule 140 may include an aptamer, wherein the aptamer is a short, single-stranded nucleic acid that can fold into unique three-dimensional structures to bind specifically to target analyte 104. In a further non-limiting example, anti-analyte molecule 140 may include a molecularly imprinted polymer (MIP), wherein the MIP is a synthetic material designed to mimic the recognition properties of biological receptors.

Still referring to FIG. 1, a “nanoparticle,” for the purpose of this disclosure, is a small particle with dimensions in the nanometer scale, typically ranging from 1 to 100 nanometers. In some cases, nanoparticle 144 may exhibit unique physical, chemical, and optical properties that are distinct from their bulk counterparts due to their high surface-to-volume ratio, quantum confinement effects, and size-dependent phenomena. In an embodiment, nanoparticle 144 may include a 40 nm gold nanoparticle (GNP or AuNP). Gold nanoparticles are widely used in biosensing and diagnostic applications due to their unique optical properties, biocompatibility, and ease of surface functionalization. In a non-limiting example, GNP may exhibit strong surface plasmon resonance (SPR) absorption and scattering, which can be tuned by controlling their size, shape, and composition. In another embodiment, nanoparticle 144 may include a magnetic nanoparticle (MNP) such as, without limitation, iron oxide nanoparticles (e.g., Fe₃O₄ or γ-Fe₂O₃). MNPs are commonly used in biosensing applications for their magnetic properties. In another embodiment, nanoparticle 144 may include a quantum dot. Quantum dots are semiconductor nanoparticles that exhibit size-dependent optical and electronic properties, including tunable fluorescence emission and high quantum yields. Other exemplary embodiments of nanoparticle 144 may include, without limitation, silica nanoparticles, carbon-based nanoparticles, and the like.

In a non-limiting example, and with further reference to FIG. 1, conjugation of anti-analyte molecules 140 with nanoparticle 144 may facilitate signal amplification, thereby enhancing the sensitivity of the analyte detection. Nanoparticle 144 is configured to provide a binding signal 148 to biosensor 108 when nanoparticle 144 binds to analyte 104 bound to binding ligand 116 on at least a sensor surface 112. As used in this disclosure, a “binding signal” is a detectable signal generated by biosensor 108 or any sensor circuit described in this disclosure when a target analyte 104 specifically interact with its corresponding biological recognition component, such as a bioreceptor, immobilized on at least a sensor surface 112 of biosensor 108.

Conjugation of anti-analyte molecule 140 with nanoparticle 144 may involve a covalent or non-covalent attachment of anti-analyte molecule 140 to nanoparticle surface, creating a functional hybrid nanostructure that combines the unique properties of nanoparticle 144 with the specific recognition capabilities of anti-analyte molecule 140. In an embodiment, conjugating anti-analyte molecule 140 with nanoparticle 144 may include functionalizing nanoparticle 144; for instance, and without limitation, the surface of nanoparticle 144 may be modified to introduce one or more functional groups that facilitate the attachment of the anti-analyte molecule 140 through various method such as, without limitation, ligand exchange, layer-by-layer assembly, or situ syntheses. In a non-limiting example, GNP may be functionalized with thiol-containing molecules, forming a self-assembled monolayer (SAM) on the gold surface via a strong gold-thiol bound. In some cases, conjugating anti-analyte molecule 140 with nanoparticle 144 may include functionalizing nanoparticle 144 may also include introducing a linker molecule to control the orientation, spacing, and accessibility of anti-analyte molecules on nanoparticle surface; for instance, and without limitation, linker molecule such as a bifunctional PEG (polyethylene glycol) derivatives, heterobifunctional crosslinkers, or specific biomolecules (e.g., streptavidin, protein A/G, and the like) containing reactive groups may be introduced, by microfluidic device 120, bind to both the surface of nanoparticle 144 and anti-analyte molecule 140. Continuing the non-limiting example, bifunctional PEG molecules containing a thiol group at one end and an amine-reactive NHS (N-hydroxy succinimide) ester group at another end may be introduced to functionalized GNP, thereby forming a gold-thiol bonds and presenting the reactive NHS easter groups on nanoparticle surface. Anti-analyte molecule 140 may be then attached to the functionalized nanoparticle surface through covalent or non-covalent interactions as a function of the linker molecule. In some cases, covalent interactions may include, without limitation, amide bond formation, disulfide bond formation, click chemistry, or the like, while non-covalent interactions may involve electrostatic, hydrophobic, or affinity-based interactions, such as biotin-streptavidin, antigen-antibody binding, or the like. Continuing the non-limiting example, an antibody specific for target analyte 104 may be conjugated to the functionalized GNP through amide bond formation between the antibody's primary amine groups (e.g., lysine residues) and the NHS easter groups on the PEG linker molecules. After conjugation, the physicochemical properties of anti-analyte molecule-nanoparticle conjugates, such as, without limitation, size, morphology, surface charge, functional group density, and/or the like may be characterized to ensure conjugates' stability, functionality, and/or performance. Additionally, or alternatively, conjugating anti-analyte molecule 140 with MNP may allow for magnetic separation, concentration, and detection of target analyte 104 in sample fluid 128.

With continued reference to FIG. 1, in some cases, conjugating anti-analyte molecule 140 with nanoparticle 144 may include transferring biosensor 108 into a second fluid containing nanoparticle 144. In a non-limiting example, biosensor 108 with analyte 104 binds to binding ligand 116 may be transferred from a first microfluidic feature such as, without limitation, functionalized biosensor region 136, to a second microfluidic feature such as, without limitation, a microfluidic chamber containing 200 pM GNP. Additionally, or alternatively, plurality of microfluidic feature 124 may include a conjugate pad, wherein the “conjugate pad,” as described herein, is a component configured to house or apply second fluid such as, without limitation, a reagent. In some cases, reagents may include a mixture of nanoparticles 144. In some embodiment, conjugate pad may be configured to provide a consistent and controlled microfluidic environment for reagents; for instance, and without limitation, by controlling the amount and rate of nanoparticle delivery. In other embodiments, conjugate pad may provide a surface or support for conjugation of anti-analyte molecule 140 with nanoparticles 144.

With continued reference to FIG. 1, in some cases, conjugating the anti-analyte molecule with the nanoparticle may include purifying sample fluid 128 as a function of the anti-analyte molecule-nanoparticle conjugates. The presence of anti-analyte molecule-nanoparticle conjugates in sample fluid 128 may selectively binding to the target analyte and facilitating the separation from other components in the sample fluid 128, thereby achieving purification of sample fluid 128. In a non-limiting example, sample fluid 128 may contain a complex mixture of molecules that may potentially interfere with biosensing process, leading to false-positive or false-negative results. Purification may ensure that only target analyte 104 is present in sample fluid 128, minimizing the risk of non-specific binding or signal interference. The presence of contaminants in sample fluid 128 may also negatively impact the functionality and stability of bioreceptors on the at least a sensor surface 112 of biosensor 108, resulting in reduced binding efficiency and signal-to-noise ratio. Purification may also ensure that sample fluid 128 is free of such contaminants, preserving the bioreceptor's functionality and enhancing the overall performance of biosensor 108. In some cases, target analyte may be present in low concentrations, making it challenging to detect using conventional biosensing techniques. Purification with anti-analyte molecule-nanoparticle conjugates may help concentrate target analyte 104, thereby improving the sensitivity and detection limit of biosensor 108. Additionally, or alternatively, purifying sample fluid 128 may include washing anti-analyte molecule-nanoparticle conjugates with suitable buffer to remove any remaining impurities or non-specifically bound molecules and resuspending purified sample fluid in buffer fluid 132.

With continued reference to FIG. 1, microfluidic device 120 is configured to incubate biosensor 108 with analyte 104 bound to binding ligand 116 on at least a sensor surface 112 with anti-analyte molecule 140 conjugated with the nanoparticle 144. In an embodiment, microfluidic device 120 may initiate a secondary binding event, which may enhance the detectable signal. In some cases, this second incubation process may enable a sandwich-type assay. In a non-limiting example, anti-analyte molecule 140 conjugated with nanoparticle 144 may serve as a secondary bioreceptor (while binding ligand may serve as a primary bioreceptor) used to capture and detect target analyte 104. Anti-analyte molecule 140 on nanoparticle 144 may specifically recognize and bind to the captured analyte (by binding ligand 116) on the at least a sensor surface 112 of biosensor 108, thereby forming a sandwich-like complex. Anti-analyte molecule 140 on nanoparticle 144 may be specific for a different epitope on target analyte 104, allowing it to bind to analyte 104 without interfering with the binding ligand-analyte interaction. Such binding may generate a stronger detectable signal as a function of nanoparticle 144 conjugated to anti-analyte molecule bound to analyte 104 captured by binding ligand on at least a sensor interface 112 of biosensor 108. Additionally, or alternatively, unbound anti-analyte molecule-nanoparticle conjugates may be removed by washing biosensor 108, reducing the potential for non-specific signals.

With continued reference to FIG. 1, Apparatus includes a sensor circuit 152. Sensor circuit 152 may include any computing device as described in this disclosure, including without limitation a microcontroller, microprocessor, digital signal processor (DSP) and/or system on a chip (SoC) as described in this disclosure. Sensor circuit 152 may also include any combinational and/or sequential logic circuit, such as, without limitation, an application-specific integrated circuit (ASIC), field-programmable gate array (FPGA), or other logic circuit. In a non-limiting example, sensor circuit 152 may be or be connected to a microcontroller, computing device, and/or the like and may be programmed using hardware and/or software design by the microcontroller and/or the computing device. Sensor circuit 152 may include, be included in, and/or communicate with a mobile device such as a mobile telephone or smartphone. Sensor circuit 152 may include a single computing device operating independently, or may include two or more computing device operating in concert, in parallel, sequentially or the like; two or more computing devices may be included together in a single computing device or in two or more computing devices. Sensor circuit 152 may interface or communicate with one or more additional devices as described below in further detail via a network interface device. Network interface device may be utilized for connecting sensor circuit 152 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Sensor circuit 152 may include but is not limited to, for example, a computing device or cluster of computing devices in a first location and a second computing device or cluster of computing devices in a second location. Sensor circuit 152 may include one or more computing devices dedicated to data storage, security, distribution of traffic for load balancing, and the like. Sensor circuit 152 may distribute one or more computing tasks as described below across a plurality of computing devices of computing device, which may operate in parallel, in series, redundantly, or in any other manner used for distribution of tasks or memory between computing devices. Sensor circuit 152 may be implemented using a “shared nothing” architecture in which data is cached at the worker, in an embodiment, this may enable scalability of apparatus 100 and/or computing device.

With continued reference to FIG. 1, sensor circuit 152 may be designed and/or configured to perform any method, method step, or sequence of method steps in any embodiment described in this disclosure, in any order and with any degree of repetition. For instance, sensor circuit 152 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Sensor circuit 152 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

With continued reference to FIG. 1, sensor circuit 152 is communicatively connected to biosensor 108. As used in this disclosure, “communicatively connected” means connected by way of a connection, attachment, or linkage between two or more relata which allows for reception and/or transmittance of information therebetween. For example, and without limitation, this connection may be wired or wireless, direct, or indirect, and between two or more components, circuits, devices, systems, and the like, which allows for reception and/or transmittance of data and/or signal(s) therebetween. Data and/or signals therebetween may include, without limitation, electrical, electromagnetic, magnetic, video, audio, radio, and microwave data and/or signals, combinations thereof, and the like, among others. A communicative connection may be achieved, for example and without limitation, through wired or wireless electronic, digital, or analog, communication, either directly or by way of one or more intervening devices or components. Further, communicative connection may include electrically coupling or connecting at least an output of one device, component, or circuit to at least an input of another device, component, or circuit. For example, and without limitation, using a bus or other facility for intercommunication between elements of a sensor circuit 154. Communicative connecting may also include indirect connections via, for example and without limitation, wireless connection, radio communication, low power wide area network, optical communication, magnetic, capacitive, or optical coupling, and the like. In some instances, the terminology “communicatively coupled” may be used in place of communicatively connected in this disclosure.

Still referring to FIG. 1, in some cases, apparatus 100 and/or biosensor 108 may perform one or more signal processing steps on a signal. For instance, apparatus 100 and/or biosensor 108 may analyze, modify, and/or synthesize a signal representative of data in order to improve the signal, for instance by improving transmission, storage efficiency, or signal to noise ratio. Exemplary methods of signal processing may include analog, continuous time, discrete, digital, nonlinear, and statistical. Analog signal processing may be performed on non-digitized or analog signals. Exemplary analog processes may include passive filters, active filters, additive mixers, integrators, delay lines, compandors, multipliers, voltage-controlled filters, voltage-controlled oscillators, and phase-locked loops. Continuous-time signal processing may be used, in some cases, to process signals which vary continuously within a domain, for instance time. Exemplary non-limiting continuous time processes may include time domain processing, frequency domain processing (Fourier transform), and complex frequency domain processing. Discrete time signal processing may be used when a signal is sampled non-continuously or at discrete time intervals (i.e., quantized in time). Analog discrete-time signal processing may process a signal using the following exemplary circuits sample and hold circuits, analog time-division multiplexers, analog delay lines and analog feedback shift registers. Digital signal processing may be used to process digitized discrete-time sampled signals. Commonly, digital signal processing may be performed by a sensor circuit or other specialized digital circuits, such as without limitation an application specific integrated circuit (ASIC), a field-programmable gate array (FPGA), or a specialized digital signal processor (DSP). Digital signal processing may be used to perform any combination of typical arithmetical operations, including fixed-point and floating-point, real-valued and complex-valued, multiplication and addition. Digital signal processing may additionally operate circular buffers and lookup tables. Further non-limiting examples of algorithms that may be performed according to digital signal processing techniques include fast Fourier transform (FFT), finite impulse response (FIR) filter, infinite impulse response (IIR) filter, and adaptive filters such as the Wiener and Kalman filters. Statistical signal processing may be used to process a signal as a random function (i.e., a stochastic process), utilizing statistical properties. For instance, in some embodiments, a signal may be modeled with a probability distribution indicating noise, which then may be used to reduce noise in a processed signal.

With continued reference to FIG. 1, sensor circuit 152 is configured to detect at least an analyte characteristic 156 of analyte 104 as a function of binding signal 148. Sensor circuit 152 may be configured to receive one or more signals from biosensor 108 such as, without limitation, binding signal 148 as described above. As used in this disclosure, an “analyte characteristic” refers to a specific property or feature of the target analyte that can be detected and measured as a result of the binding signal 148 generated by biosensor 108. In an embodiment, analyte characteristic 156 may be associated with the interaction between analyte 104 and binding ligand 116 immobilized on at least a sensor surface 112, leading to a detectable change or response in biosensor 108. In another embodiment, analyte characteristic 156 may be associated with the interaction between anti-analyte molecule-nanoparticle conjugates and analyte 104 bound to binding ligand 116 immobilized on at least a sensor surface 112. In some cases, analyte characteristics 156 may be used to identify, quantify, or monitor the presence of the target analyte 104 in sample fluid 128. In a non-limiting example, analyte characteristic 156 may include an analyte concentration, wherein the analyte concentration is the amount or relative abundance of target analyte 104 in sample fluid 128. Binding signal 148 generated by biosensor 108 may be directly proportional to the analyte concentration, enabling its quantification. In another non-limiting example, analyte characteristic 156 may include a binding affinity, wherein the binding affinity is the strength of the interaction between target analyte 104 and bioreceptor on at least a sensor surface 112 of biosensor 108. Sensor circuit 152 may determine binding affinity by analyzing binding signal as a function of time or analyte concentration of the interaction. Binding affinity may be useful in characterizing target analyte 104 or its interaction with other molecules. Other exemplary embodiments of analyte characteristics 156 may include, without limitation, molecular size, molecular structure, analyte specificity, and the like. Persons skilled in the art, upon reviewing the entirety of this disclosure, may appreciate various analyte characteristics 156 that may be detected by sensor circuit 152 of apparatus 100. Additionally, sensor circuit 152 may facilitate real-time monitoring, data storage, and sharing of analyte characteristics 156, enabling more advanced data analysis and decision-making in multiplexed and point-of-care diagnostic of analytes 104.

With continued reference to FIG. 1, in some cases, detecting at least an analyte characteristic 156 may include utilizing a biolayer interferometry (BLI) system 160 to monitor an interaction between analyte 104 and binding ligand 116 in real time. A “Biolayer interferometry system,” for the purpose of this disclosure, is an optical biosensing technology used to study molecular interactions. In a non-limiting example, sensor circuit 152 may be integrated with BLI system 160 to monitor the interaction between analyte 104 and binding ligand/anti-analyte molecule nanoparticle conjugates in real time. BLI system 160 may measure changes in the interference pattern of light reflected from at least a sensor surface 112 as molecule such as, without limitation, analyte 104, bind or dissociate from at least a sensor surface 112. Such real-time, label-free techniques may provide valuable information on analyte characteristics 156 of analyte 104 as described above. As described herein, an “interference pattern” is a phenomenon in optics that occurs when two or more coherent light waves overlap, resulting in a spatial distribution of light intensity due to constructive and destructive interference. In some cases, a change in the interference pattern may be caused by alterations in the optical path length or the thickness of the biolayer (i.e., layer of binding ligand) on at least a sensor surface 112. When analyte 104 bind to or dissociates from at least a sensor surface, the thickness and density of biolayer may change, which in turn affects the optical path length of the light waves reflecting off at least a sensor surface 112. As the optical path length changes, the phase relationship between the reflected light waves may also change, leading to a shift in the interference pattern. Such shift may be directly related to the amount of analyte 104 bound to or dissociated from at least a sensor surface 112, allowing BLI system 160 to monitor molecular interactions in real time. In some cases, binding signal 148 may include one or more changes in the interference pattern. Such binding signal 148 may represent a real-time interaction between analyte 104 and binding ligand 116 and/or anti-analyte molecule nanoparticle conjugates. Sensor circuit 152 may analyze binding signal 148 over time to determine at least an analyte characteristic 156 such as, without limitation, analyte concentration of target analyte 104.

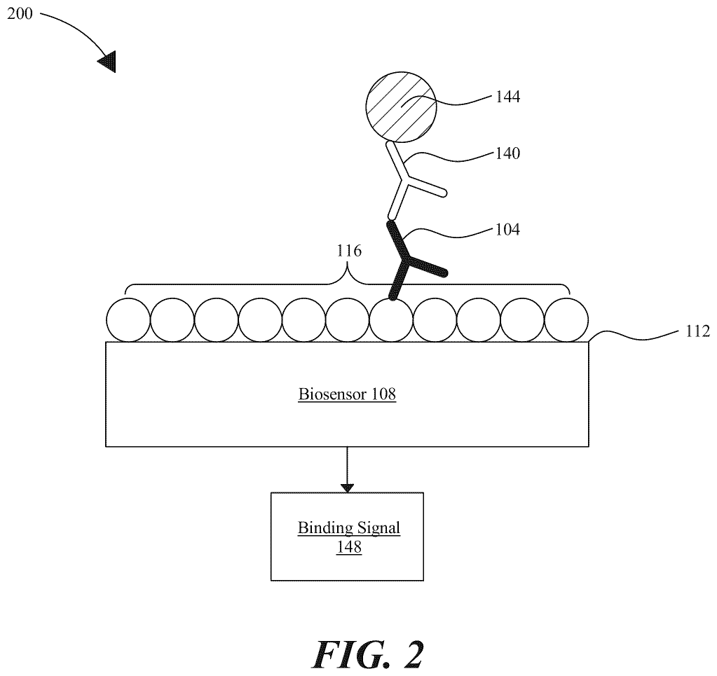

Now referring to FIG. 2, an example of an assay 200 is illustrated. Assay 200 may include a sandwich-type assay as described above with reference to FIG. 1. Biosensor 108 may be functionalized with AraH2 protein (i.e., binding ligand 116). Biosensor 108 may be incubated with sample fluid 128 containing anti-AraH2 antibodies (i.e., analyte 104), which bind to at least a sensor surface 112. An anti-IgE antibody (i.e., anti-analyte molecule 140) may conjugate to a 40 nm GNP (i.e., nanoparticle 144) which may give a strong binding signal 148 when (anti-IgE antibody) bound to (anti-AraH2 antibody bind to AraHe protein immobilized on) at least a sensor surface 112, thereby forming a sandwich-like complex 204. In some cases, Attachment of GNPs binding may be monitored using BLI system 160 as described above with reference to FIG. 1 such as, without limitation, a GATORBIO.

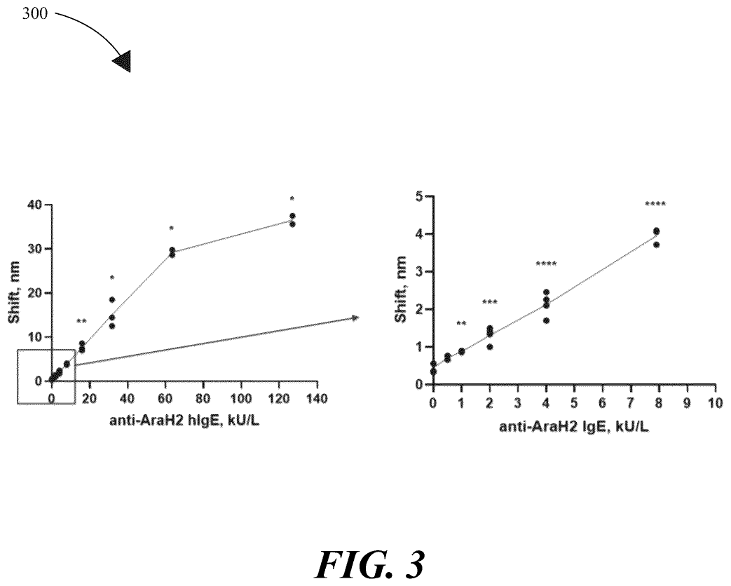

Now referring to FIG. 3, an exemplary assay dynamic range 300 is illustrated. As used in this disclosure, an “assay dynamic range” refers to the range of concentrations of analyte 104 within which a specific assay can accurately and reliably measure or detect analyte 104. In a non-limiting example, assay dynamic range 300 may be a span between a lower limit of detection (LOD) and an upper limit of quantitation (ULOQ) for the assay. A “lower limit of detection (LOD),” for the purpose of this disclosure, is the smallest concentration of analyte 104 that can be reliably detected and distinguished from the background signal or noise. An “upper limit of quantitation (ULOQ),” for the purpose of this disclosure, is the highest concentration of analyte 104 at which the assay can accurately measure the analyte without encountering saturation, nonlinearity, or other technical limitations. In an embodiment, a wide dynamic range may be desirable for an assay, as it allows the measurement of analyte concentrations over a broader range, making the assay more versatile and applicable for different sample fluids and experimental conditions. Assays with a wide dynamic range may accurately quantify both low-abundance and high-abundance analytes within the same sample fluid, simplifying the analysis and reducing the need for sample dilution or concentration adjustments. In a non-limiting example, assay dynamic range 300 of assay 200 described above may be characterized in PBS/EDTA/Tween-20 buffer spiked with 0-125 kU/L of monoclonal human anti-AraH2 IgE. Biosensor 108 may be functionalized with AraH2 protein and incubated in the buffer spiked with 0-125 kU/L of monoclonal human anti-AraH2 IgE. After the incubation, biosensor 108 may be transferred to 200 pM GNP and GNP binding may be measured using GATORBIO. Assay dynamic range 300 of 1-125 kU/L of anti-AraH2 IgE may be detected using such assay set up.

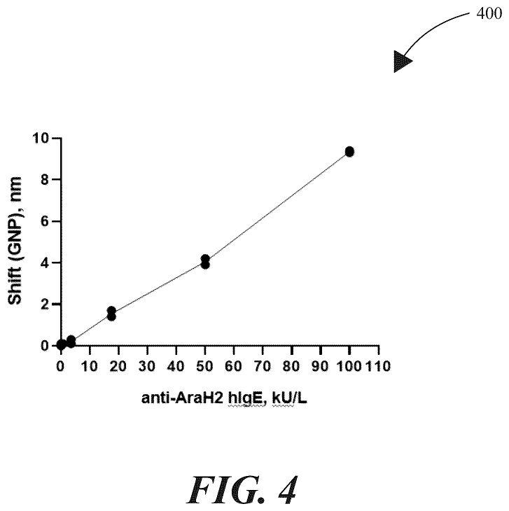

Now referring to FIG. 4, an exemplary embodiment 400 of a signal decrease in presence of 50% serum is illustrated. Biosensor 108 functionalized with AraH2 protein may be incubated in a 50% human Ig-free serum/PBS/EDTA/Tween-20/Zwittergent3-14 buffer spiked with 0-125 kU/L of monoclonal human anti-AraH2 IgE. Biosensor 108 may then be transferred into 200 pM GNP in the same buffer but without IgE and GNP binding may be analyzed using GATORBIO. As follows from FIG. 4, presence of serum proteins may result in approximately 4-fold reduction of the signal (i.e., binding signal 148). 100 kU/L of anti-AraH2 IgE in buffer led to 35 nm shift after adding GNP (as shown in FIG. 2), while in 50% plasma the same concentration of sIgE induced only 10 nm shift.

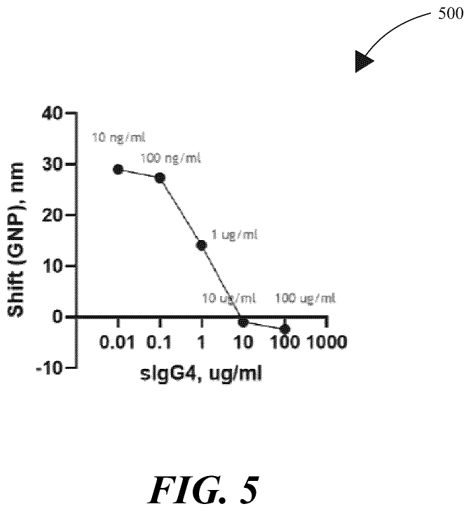

Now referring to FIG. 5, an exemplary embodiment 500 of a sIgG binding is illustrated. Binding of sIgG to at least a sensor surface 112 of biosensor 108 may perturb sIgE quantification. In a non-limiting example, biosensor 108 may be functionalized with AraH2 protein and incubated in PBS/EDTA/Tween-20 buffer spiked with 100 kU/L of monoclonal human anti-AraH2 IgE and 0-100 μg/ml of monoclonal human anti-AraH2 IgG4 targeting the same epitope as the IgE. Biosensor 108 may then be transferred to 200 pM GNP and GNP binding to at least a sensor surface 112 may be measured using GATORBIO. As shown in FIG. 5, IgG4 concentrations may be higher than 100 ng/ml resulting in a noticeable decrease in GNP binding to biosensor 108, thereby perturbing quantification of anti-AraH2 IgE concentration. Based on literature data, both sIgG and sIgG4 may be higher than sIgE, ratios at the highest concentration are 1,000 for sIgG/sIgE, and ˜150 for sIgG4/sIgE; therefore, for clinically meaningful sIgE levels of 0.35-100 kU/L (0.8-244 ng/ml), sIgG concentration may be 0.128-36.6 ug/ml, which is much higher than the minimum in assay 200 (100 ng/ml).

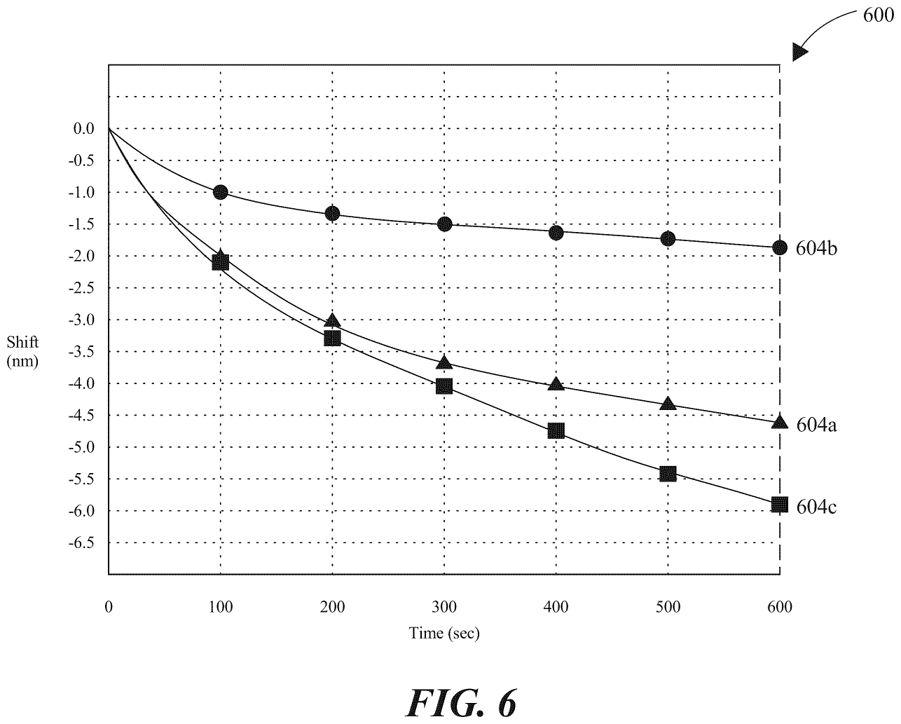

Now referring to FIG. 6, an exemplary embodiment 600 of improvement to sIgE detection using IgE concentration using anti-IgE MNP. In some cases, IgE concentration using anti-IgE MNP may be used to improve sIgE detection. In a non-limiting example, three sample fluid 604a-c using 269 pM of anti-IgE antibody conjugated to 100 nm magnetic beads (MNP) may be prepared by microfluidic device 120. First Sample fluid 604a may include 50% Ig-free serum/PBS/EDTA/Tween-20 spiked with 17.5 kU/L of monoclonal human anti-AraH2 IgE and 1000 kU/L of polyclonal human IgE. Second sample fluid 604b may include 50% Ig-free serum/PBS/EDTA/Tween-20 spiked with 25 μg/ml of monoclonal anti-AraH2 IgG4 targeting the same epitope as the anti-AraH2 IgE and 7.5 mg/ml of polyclonal human IgG. Third sample fluid 604c may include 50% Ig-free serum/PBS/EDTA/Tween-20 spiked with 17.5 kU/L of monoclonal human anti-AraH2 IgE, 1000 kU/L of polyclonal human IgE, 25 μg/ml of monoclonal anti-AraH2 IgG4 targeting the same epitope as the anti-AraH2 IgE and 7.5 mg/ml of polyclonal human IgG. Purified IgE-MNP complexes may be resuspended in buffer. MNP binding to biosensor 108 functionalized with AraH2 may be tested using GATORBIO. First sample fluid 604a and third sample fluid 604c containing sIgE produced 2.5-3 times higher shift compared to the blank (i.e., second sample fluid 604b). It is to be noted that the approach described herein for IgEs may be used for any analyte disclosed or undisclosed herein.

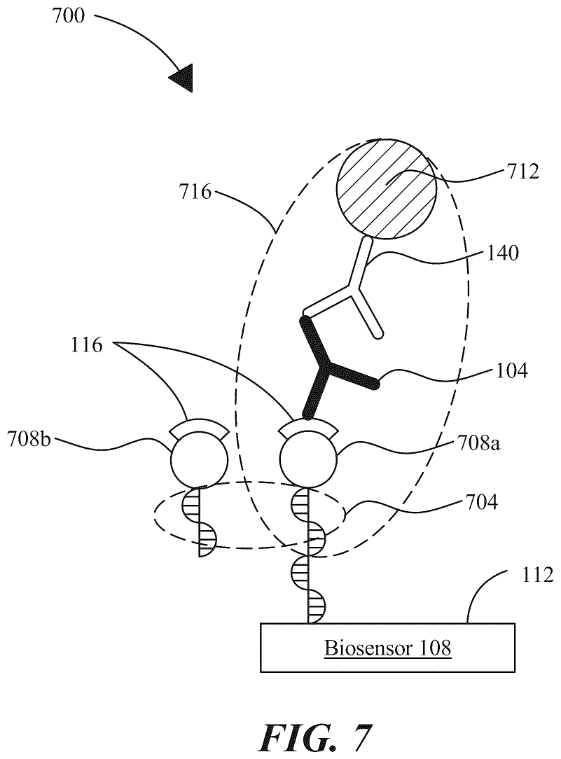

Now referring to FIG. 7A, an exemplary embodiment 700 of a sandwich-type assay using magnetic beads is illustrated. IgE testing may be beneficial to take a barcoded approach. In an embodiment, at least a sensor surface 112 may be functionalized with a barcode 704. A “barcode,” for the purpose of this disclosure, refers to a unique pattern or arrangement of biomolecules. In a non-limiting example, barcode 704 may include, without limitation, DNA, proteins, peptides, or the like. In some cases, barcode 704 may serve as a bioreceptor (i.e., primary bioreceptor) as described above on at least a sensor surface 112, enabling the detection of multiple analytes 104 simultaneously in a multiplexed format. In some cases, particles 708a-b coated in a capture (i.e., binding ligand 116) may be added to sample fluid 128 alongside a plurality of magnetic beads 712. In a non-limiting example, particles 708 may include nanoparticles such as GNPs as described above. In some cases, magnetic beads may also be barcoded. This may allow a sandwich complex 716 to form between magnetic bead 712 and one of the capture-coated particles 708a-b such that when the wash is done only the analyte 104 that are bound to magnetic bead 712 are left. When the washed sample flows over biosensor 108, the sandwich complex 716 may bind to biosensor 108 that matches their barcodes. Biosensor 108 may include a plurality of ring resonators as described above. In some cases, taking such barcoded approach may allow a three-dimensional (3D) reaction. As used in this disclosure, a “3D reaction” refers to a more effective interaction between analyte 104 and functionalized sensor surface due to a 3D arrangement of barcode 704.

In a non-limiting example, and still referring to FIG. 7, 3D arrangement of barcode 704 on at least a sensor surface 112 may be the use of DNA-origami nanostructures for multiplexed detection, wherein the DNA origami is a technique that allows the folding of long single-stranded DNA molecules into custom-designed 3D shapes by utilizing a set of short DNA strands, called staple strands, to hold the structure together. These DNA-origami nanostructures may serve as 3D barcodes on at least a sensor surface 112, providing a versatile platform for detecting various target analytes 104. In some cases, DNA-origami nanostructure may be designed to have multiple binding ligands 116, such as DNA aptamers or protein-binding peptides, attached to its surface in a specific 3D arrangement. These binding ligands 116 may serve as bioreceptors for different target analytes. Such DNA-origami nanostructure may be then immobilized onto at least a sensor surface 112, thereby creating a 3D barcode that can specifically recognize and bind to multiple analytes.

Additionally, or alternatively, with further reference to FIG. 7, 3D reaction may give better kinetics, providing more binding surface area. Larger binding surface area may be important in assays such as assay 200 as described above where the binding ligand 116 may be a mixture of many antigens with different abundances. In other cases, barcoded approach may also allow biosensor 108 to be generic to any assay just by switching out the detection particle upstream with a particle that has the same barcode but a different capture. It is to be noted that the barcoded approach described herein for IgEs may be used for any analyte.

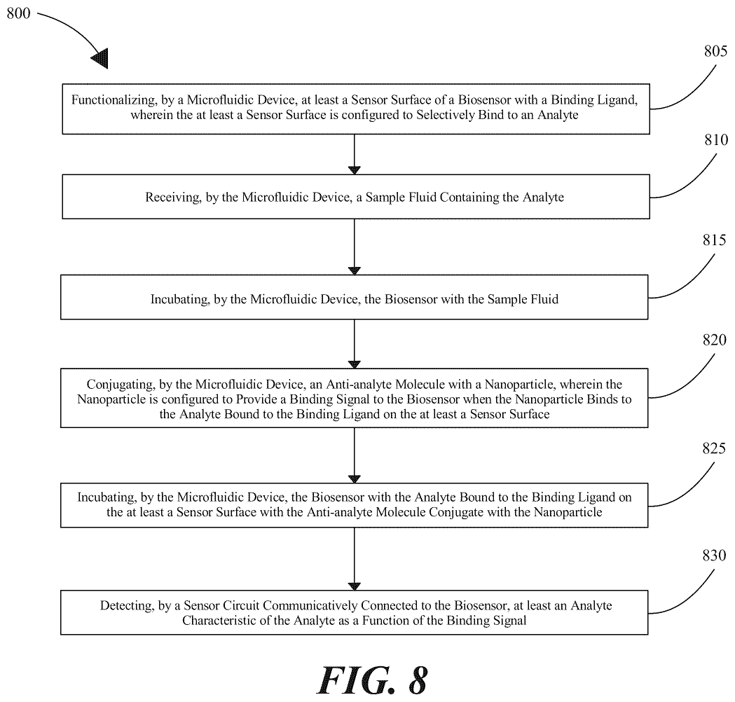

Now referring to FIG. 8, a flow diagram of an exemplary method 800 for detecting an analyte is illustrated. Method 800 includes a step 805 of functionalizing, by a microfluidic device, at least a sensor surface of a biosensor with a binding ligand, wherein the at least a sensor surface is configured to selectively bind to an analyte. In some embodiments, the at least a sensor surface of the biosensor may be functionalized with a barcode. This may be implemented, without limitation, as described above with reference to FIGS. 1-7.

With continued reference to FIG. 8, method 800 includes a step 810 of receiving, by the microfluidic device, a sample fluid containing the analyte. In some embodiments, receiving the sample fluid may include spiking a buffer fluid. This may be implemented, without limitation, as described above with reference to FIGS. 1-7.

With continued reference to FIG. 8, method 800 includes a step 815 of incubating, by the microfluidic device, the biosensor with the sample fluid. This may be implemented, without limitation, as described above with reference to FIGS. 1-7.

With continued reference to FIG. 8, method 800 includes a step 820 of conjugating, by the microfluidic device, an anti-analyte molecule with a nanoparticle, wherein the nanoparticle is configured to provide a binding signal to the biosensor when the nanoparticle binds to the analyte bound to the binding ligand on the at least a sensor surface. In some embodiments, the nanoparticle may include a gold nanoparticle (GNP). In some embodiments, the nanoparticle may include a magnetic bead nanoparticle (MNP). In some embodiments, conjugating the anti-analyte molecule with the nanoparticle may include transferring the biosensor into a second fluid containing the nanoparticle. In some embodiments, conjugating the anti-analyte molecule with the nanoparticle may include purifying the sample fluid as a function of the conjugates. In some embodiments, purifying the sample fluid may include resuspending purified sample fluid in a buffer fluid. This may be implemented, without limitation, as described above with reference to FIGS. 1-7.

With continued reference to FIG. 8, method 800 includes a step 825 of incubating, by the microfluidic device, the biosensor with the analyte bound to the binding ligand on the at least a sensor surface with the anti-analyte molecule conjugated with the nanoparticle. This may be implemented, without limitation, as described above with reference to FIGS. 1-7.

With continued reference to FIG. 8, method 800 includes a step 830 of detecting, by a sensor circuit communicatively connected to the biosensor, at least an analyte characteristic of the analyte as a function of the binding signal. In some embodiments, the biosensor may be configured to enable a three-dimensional (3D) reaction as a function of the barcode. In some embodiments, detecting at least an analyte characteristic may include utilizing a biolayer interferometry (BLI) system to monitor an interaction between the analyte and the binding ligand in real time. This may be implemented, without limitation, as described above with reference to FIGS. 1-7.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding can readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

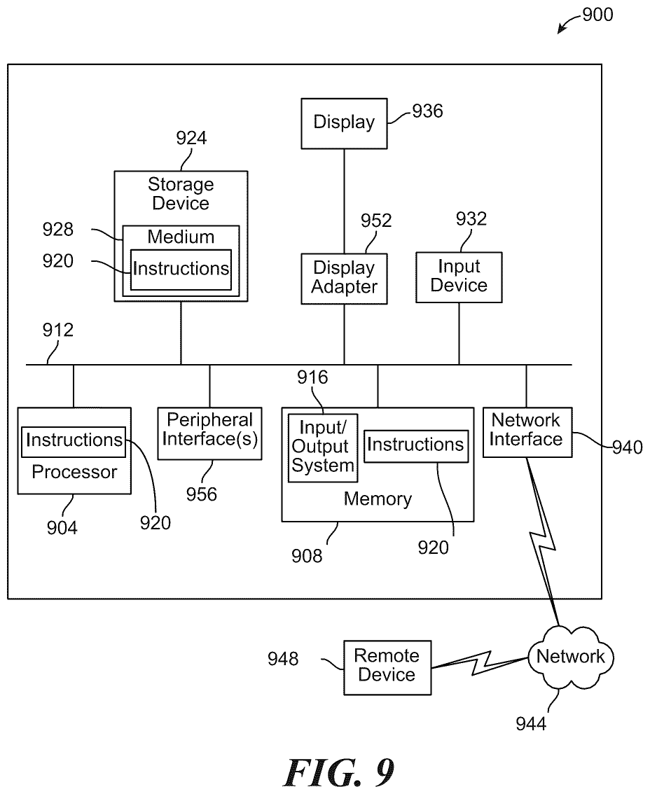

FIG. 9 shows a diagrammatic representation of one embodiment of a computing device in the exemplary form of a computer system 900 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 900 includes a processor 904 and a memory 908 that communicate with each other, and with other components, via a bus 912. Bus 912 may include any of several types of bus structures including, but not limited to, a memory bus, a memory controller, a peripheral bus, a local bus, and any combinations thereof, using any of a variety of bus architectures.

Processor 904 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 904 may be organized according to Von Neumann and/or Harvard architecture as a non-limiting example. Processor 904 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 908 may include various components (e.g., machine-readable media) including, but not limited to, a random-access memory component, a read only component, and any combinations thereof. In one example, a basic input/output system 916 (BIOS), including basic routines that help to transfer information between elements within computer system 900, such as during start-up, may be stored in memory 908. Memory 908 may also include (e.g., stored on one or more machine-readable media) instructions (e.g., software) 920 embodying any one or more of the aspects and/or methodologies of the present disclosure. In another example, memory 908 may further include any number of program modules including, but not limited to, an operating system, one or more application programs, other program modules, program data, and any combinations thereof.

Computer system 900 may also include a storage device 924. Examples of a storage device (e.g., storage device 924) include, but are not limited to, a hard disk drive, a magnetic disk drive, an optical disc drive in combination with an optical medium, a solid-state memory device, and any combinations thereof. Storage device 924 may be connected to bus 912 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 924 (or one or more components thereof) may be removably interfaced with computer system 900 (e.g., via an external port connector (not shown)). Particularly, storage device 924 and an associated machine-readable medium 928 may provide nonvolatile and/or volatile storage of machine-readable instructions, data structures, program modules, and/or other data for computer system 900. In one example, software 920 may reside, completely or partially, within machine-readable medium 928. In another example, software 920 may reside, completely or partially, within processor 904.

Computer system 900 may also include an input device 932. In one example, a user of computer system 900 may enter commands and/or other information into computer system 900 via input device 932. Examples of an input device 932 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 932 may be interfaced to bus 912 via any of a variety of interfaces (not shown) including, but not limited to, a serial interface, a parallel interface, a game port, a USB interface, a FIREWIRE interface, a direct interface to bus 912, and any combinations thereof. Input device 932 may include a touch screen interface that may be a part of or separate from display 936, discussed further below. Input device 932 may be utilized as a user selection device for selecting one or more graphical representations in a graphical interface as described above.

A user may also input commands and/or other information to computer system 900 via storage device 924 (e.g., a removable disk drive, a flash drive, etc.) and/or network interface device 940. A network interface device, such as network interface device 940, may be utilized for connecting computer system 900 to one or more of a variety of networks, such as network 944, and one or more remote devices 948 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 944, may employ a wired and/or a wireless mode of communication. In general, any network topology may be used. Information (e.g., data, software 920, etc.) may be communicated to and/or from computer system 900 via network interface device 940.

Computer system 900 may further include a video display adapter 952 for communicating a displayable image to a display device, such as display device 936. Examples of a display device include, but are not limited to, a liquid crystal display (LCD), a cathode ray tube (CRT), a plasma display, a light emitting diode (LED) display, and any combinations thereof. Display adapter 952 and display device 936 may be utilized in combination with processor 904 to provide graphical representations of aspects of the present disclosure. In addition to a display device, computer system 900 may include one or more other peripheral output devices including, but not limited to, an audio speaker, a printer, and any combinations thereof. Such peripheral output devices may be connected to bus 912 via a peripheral interface 956. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions, and additions may be made to that which is specifically disclosed herein without departing from the spirit and scope of the present invention.

What is claimed is:

1. An apparatus for detecting an analyte, the apparatus comprises:
a biosensor, wherein the biosensor comprises: at least a sensor surface functionalized with a binding ligand, wherein the at least a sensor surface is configured to selectively bind to an analyte;
a microfluidic device, wherein the microfluidic device is configured to:
receive a sample fluid containing the analyte;
incubate the biosensor with the sample fluid; and
conjugate an anti-analyte molecule with a nanoparticle, wherein the nanoparticle is configured to provide a binding signal to the biosensor when the nanoparticle binds to the analyte bound to the binding ligand on the at least a sensor surface; and
incubate the biosensor with the analyte bound to the binding ligand on the at least a sensor surface with the anti-analyte molecule conjugated with the nanoparticle; and
a sensor circuit communicatively connected to the biosensor, wherein the sensor circuit is configured to:
detect at least an analyte characteristic of the analyte as a function of the binding signal.

2. The apparatus of claim 1, wherein receiving the sample fluid comprises spiking a buffer fluid with the analyte.

3. The apparatus of claim 1, wherein the nanoparticle comprises a gold nanoparticle (GNP).

4. The apparatus of claim 1, wherein the nanoparticle comprises a magnetic bead nanoparticle (MNP).

5. The apparatus of claim 1, wherein the conjugating the anti-analyte molecule with the nanoparticle comprises transferring the biosensor into a second fluid containing the nanoparticle.

6. The apparatus of claim 1, wherein the conjugating of the anti-analyte molecule with the nanoparticle comprises purifying the sample fluid as a function of the conjugates.

7. The apparatus of claim 6, wherein purifying the sample fluid comprises resuspending purified sample fluid in a buffer fluid.

8. The apparatus of claim 1, wherein the at least a sensor surface of the biosensor is functionalized with a barcode.

9. The apparatus of claim 8, wherein the biosensor is configured to enable a three-dimensional (3D) reaction as a function of the barcode.

10. The apparatus of claim 1, wherein detecting at least an analyte characteristic comprises:
utilizing a biolayer interferometry (BLI) system to monitor an interaction between the analyte and the binding ligand in real time.

11. A method for detecting an analyte, the method comprises:
functionalizing, by a microfluidic device, at least a sensor surface of a biosensor with a binding ligand, wherein the at least a sensor surface is configured to selectively bind to an analyte;
receiving, by the microfluidic device, a sample fluid containing the analyte;
incubating, by the microfluidic device, the biosensor with the sample fluid;
conjugating, by the microfluidic device, an anti-analyte molecule with a nanoparticle, wherein the nanoparticle is configured to:
provide a binding signal to the biosensor when the nanoparticle binds to the analyte bound to the binding ligand on the at least a sensor surface;
incubating, by the microfluidic device, the biosensor with the analyte bound to the binding ligand on the at least a sensor surface with the anti-analyte molecule conjugated with the nanoparticle; and
detecting, by a sensor circuit communicatively connected to the biosensor, at least an analyte characteristic of the analyte as a function of the binding signal.

12. The method of claim 11, wherein receiving the sample fluid comprises spiking a buffer fluid with an analyte.

13. The method of claim 11, wherein the nanoparticle comprises a gold nanoparticle (GNP).

14. The method of claim 11, wherein the nanoparticle comprises a magnetic bead nanoparticle (MNP).

15. The method of claim 11, wherein the conjugating the anti-analyte molecule with the nanoparticle comprises transferring the biosensor into a second fluid containing the nanoparticle.

16. The method of claim 11, wherein the conjugating the anti-analyte molecule with the nanoparticle comprises purifying the sample fluid as a function of the conjugates.

17. The method of claim 16, wherein purifying the sample fluid comprises resuspending purified sample fluid in a buffer fluid.

18. The method of claim 11, wherein the at least a sensor surface of the biosensor is functionalized with a barcode.

19. The method of claim 18, wherein the biosensor is configured to enable a three-dimensional (3D) reaction as a function of the barcode.

20. The method of claim 11, wherein detecting at least an analyte characteristic comprises:
utilizing a biolayer interferometry (BLI) system to monitor an interaction between the analyte and the binding ligand in real time.
